(19) United States
(12) Patent Application Publication
(10) Pub. No.: US 2026/0248442 A1
(43) Pub. Date: Aug. 27, 2026

(54) PHYSIOLOGIC RESPONSE MONITOR MOUTH WEARABLE

(71) Applicant: UNIVERSITY OF SOUTH FLORIDA, Tampa, FL (US)

(72) Inventor: John Templeton, Tampa, FL (US)

(21) Appl. No.: 19/548,817

(22) Filed: Feb. 24, 2026

Related U.S. Application Data
(60) Provisional application No. 63/762,441, filed on Feb. 24, 2025.

(51) Int. Cl. A61B 5/00; A61B 5/01; A61B 5/0205; A61B 5/024; A61B 5/1455; G16H 20/00; G16H 50/30

(52) U.S. Cl. CPC A61B 5/4064; A61B 5/01; A61B 5/02055; A61B 5/02427; A61B 5/14552; A61B 5/682; A61B 5/683; A61B 5/7435; A61B 2560/0214; A61B 2560/0468; A61B 2562/0219; G16H 20/00; G16H 50/30

(57) ABSTRACT

Various components, methods, and systems are provided herein for monitoring physiological responses associated with force-impact events using a mouth-wearable sensing device positioned within an oral cavity of a subject. In some embodiments, the device integrates inertial sensing, force sensing, temperature sensing, and optical physiological sensing on a compact intraoral substrate and transmits physiological data wirelessly to an external computing device for analysis and reporting. In some embodiments, the mouth-wearable sensing device is customized using intraoral scan data to generate a three-dimensional oral cavity model and to position one or more sensors to align with anatomical landmarks when worn.

### CROSS-REFERENCE TO RELATED APPLICATIONS

This application claims priority to U.S. provisional patent application Nos. 63/762,441 filed Feb. 24, 2025, the entire contents of which are incorporated herein by reference.

### STATEMENT REGARDING FEDERALLY SPONSORED RESEARCH

N/A

### BACKGROUND

Traumatic brain injury (TBI) is a significant public health concern given its high incidence and wide-ranging effects across individuals. TBI can affect diverse populations regardless of age, sex, or socioeconomic status, including injuries arising from motor vehicle accidents, falls, sports-related impacts, and blast exposures, and the clinical presentation may vary substantially in onset and severity. As a result of this prevalence and variability, existing screening and diagnostic tools for detecting acute and early-stage TBI may exhibit insufficient sensitivity and specificity, which can contribute to under-detection or delayed intervention.

Accordingly, there is a need for technological solutions that enable objective assessment of physiological biomarkers associated with both pre-impact phases (e.g., physiological preparation) and post-impact phases (e.g., physiological response) to support more precise and personalized approaches to TBI management. While some approaches have been proposed for measuring forces undergone during head impact events, existing approaches have not provided a system capable of detecting this information in conjunction with real-time postural control across both pre-impact and post-impact phases of response. In view of these limitations, the present disclosure relates to an advanced sensing system implemented as a mouth-wearable sensing device with updated sensor deployments and hardware optimizations to support real-time assessment of postural control and autonomic function in connection with head-impact events, thereby enabling authorized personnel (e.g., coaches, athletic trainers, medical providers, caregivers, and/or command personnel) to monitor safety and make data-driven decisions, including decisions related to return-to-play or return-to-duty.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In some aspects, the present disclosure provides a mouth-wearable sensing device to be positioned within an oral cavity of a subject. In some implementations, the mouth-wearable sensing device comprises a structure to register against a roof of a mouth of the subject and maintain a fixed placement of a first sensor and a second sensor relative to the roof of the mouth when the mouth-wearable sensing device is worn. In some implementations, the first sensor is supported by the structure and positioned proximate to an incisive papilla of the subject when the mouth-wearable sensing device is worn. In some implementations, the second sensor is supported by the structure and positioned proximate to a greater palatine vasculature of the subject when the mouth-wearable sensing device is worn.

In some implementations, the structure comprises a body shaped to fit over a plurality of teeth of the subject. In some implementations, the mouth-wearable sensing device further comprises a first force sensing resistor supported by the structure and positioned in a first molar region of the oral cavity to sense a bite force of the subject when the mouth-wearable sensing device is worn, and a second force sensing resistor supported by the structure and positioned in a second molar region of the oral cavity to sense the bite force of the subject when the mouth-wearable sensing device is worn.

In some implementations, the second sensor is positioned proximate to a first branch of the greater palatine vasculature, the first sensor is a pulse oximeter, and the second sensor is a first thermistor. In some implementations, the mouth-wearable sensing device further comprises a third sensor supported by the structure and positioned proximate to a second branch of the greater palatine vasculature of the subject when the mouth-wearable sensing device is worn, the third sensor being a second thermistor.

In some implementations, the mouth-wearable sensing device further comprises a first inertial measurement unit (IMU) positioned on a first side of the oral cavity proximate to the first branch of the greater palatine vasculature when the mouth-wearable sensing device is worn, and a second inertial measurement unit (IMU) positioned on a second side of the oral cavity proximate to the second branch of the greater palatine vasculature when the mouth-wearable sensing device is worn. In some implementations, the first IMU is mounted on a first sensor assembly with the second sensor, and the second IMU is mounted on a second sensor assembly with the third sensor. In some implementations, the mouth-wearable sensing device further comprises a first accelerometer mounted on the first sensor assembly and a second accelerometer mounted on the second sensor assembly.

In some implementations, the mouth-wearable sensing device further comprises at least one sensor selected from the group consisting of a salivary sensor, a humidity sensor, a carbon dioxide (CO2) sensor, and a chemical sensor array, wherein the at least one sensor is mounted on a sensor assembly positioned in the oral cavity when the mouth-wearable sensing device is worn.

In some implementations, the mouth-wearable sensing device further comprises a wireless transmitter to transmit sensor data obtained from one or more sensors of the mouth-wearable sensing device to an external device. In some implementations, the mouth-wearable sensing device further comprises a processor to perform on-device edge processing of the sensor data prior to transmission by the wireless transmitter, wherein the wireless transmitter is further configured to stream the sensor data to a connected external device in real time.

In some aspects, the present disclosure provides a method of manufacturing a mouth-wearable sensing device to be positioned within an oral cavity of a subject. In some implementations, the method comprises obtaining an intraoral scan of the oral cavity of the subject, identifying from the intraoral characterization data a first position corresponding to an incisive papilla of the subject and a second position corresponding to a first branch of a greater palatine vasculature of the subject, and manufacturing a structure comprising a first sensor and a second sensor, the structure configured to, when worn, register against a roof of a mouth of the subject and maintain a fixed placement of the first sensor and the second sensor relative to the roof of the mouth, with the first sensor positioned proximate to the incisive papilla and the second sensor positioned proximate to the first branch of the greater palatine vasculature based on the identified positions. In some implementations, the method further comprises identifying a first molar position and a second molar position corresponding to locations of bite force concentration in the oral cavity, and manufacturing the structure to further comprise a first force sensing resistor and a second force sensing resistor positioned at those locations. In some implementations, the method further comprises identifying a third position corresponding to a second branch of the greater palatine vasculature and manufacturing the structure to further comprise a third sensor positioned proximate to the second branch of the greater palatine vasculature when worn. In some implementations, the method further comprises manufacturing the structure as a mouth guard comprising a first inertial measurement unit and a first accelerometer each mounted on a first sensor assembly with the second sensor, and a second inertial measurement unit and a second accelerometer each mounted on a second sensor assembly with the third sensor.

In some aspects, the present disclosure provides a system comprising a mouth guard to be positioned within an oral cavity of a subject and an external device. In some implementations, the mouth guard comprises a structure to register against a roof of a mouth of the subject and maintain a fixed placement of a first sensor, a second sensor, and a third sensor relative to the roof of the mouth when the mouth guard is worn, with the first sensor positioned proximate to an incisive papilla of the subject, the second sensor positioned proximate to a first branch of a greater palatine vasculature of the subject, and the third sensor positioned proximate to a second branch of the greater palatine vasculature of the subject. In some implementations, the mouth guard further comprises a wireless transmitter to transmit sensor data obtained from one or more of the first sensor, the second sensor, and the third sensor to the external device. In some implementations, the external device is to receive the sensor data and to process the received sensor data to determine whether the subject has sustained a concussion.

In some implementations, the present disclosure provides a system comprising a mouth-wearable sensing device with a plurality of sensors and a wireless transmitter, and an external device configured to receive a time-aligned multi-modal sensor data stream from the mouth-wearable sensing device and process the stream using one or more trained machine learning models to generate a severity metric representative of a force impact event experienced by the subject, and to generate, based on the severity metric, a behavioral recommendation regarding return-to-play, return-to-duty, or a recommendation to seek further medical evaluation. In some implementations, the one or more trained machine learning models comprise time-series models trained on sensor-fusion inputs combining two or more of inertial data, bite-force data, temperature data, and optical physiological data, and are configured to detect patterns and correlations across sensor modalities associated with force impact events.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments, it should be understood that such example embodiments can be implemented in various devices, systems, and methods.

### BRIEF DESCRIPTION OF THE DRAWINGS

The following figures are provided for purposes of illustration and represent non-limiting implementations of the disclosure.

FIG. 1 is a schematic plan view of an example mouth-wearable sensing device.

FIG. 2 is a perspective view of an example mouth-wearable sensing device.

FIGS. 3A-3C are perspective views illustrating examples of a mouth-wearable sensing device in a body formed of a biocompatible material.

FIG. 4 is a block diagram illustrating an example electrical architecture of the mouth-wearable sensing device, including example sensors and supporting electronics.

FIG. 5 is a block diagram conceptually illustrating a system for evaluating one or more physiological metrics using a mouth-wearable sensing device.

FIG. 6 is a flowchart illustrating an example process of training a machine learning model.

FIG. 7 is a flowchart illustrating an example process of obtaining a behavioral recommendation in response to a forceful event.

FIG. 8 is sample user interface for a Bluetooth Low Energy pairing module.

FIG. 9 is a sample user interface for a mobile companion app dashboard.

FIG. 10 is a flowchart illustrating an example process of manufacturing a mouth-wearable sensing device.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, certain known structures and components are shown in block diagram form to avoid obscuring such concepts.

In various implementations, the mouth-wearable sensing device 100 is configured to be positioned within an oral cavity of a subject and to register against a roof of the mouth of the subject when worn. As used herein, the term ‘mouth-wearable sensing device’ refers broadly to any intraoral appliance configured to be worn within the oral cavity of a subject and to support one or more sensors in a fixed positional relationship to one or more anatomical landmarks of the oral cavity. In some implementations, the mouth-wearable sensing device 100 is implemented as a mouth guard comprising a body shaped to fit over one or more teeth of the subject. In other implementations, the mouth-wearable sensing device 100 is implemented as, or incorporated into, a dental retainer, a full or partial denture, an orthodontic appliance, a palatal expander, or another intraoral appliance suitable for sustained wear, etc. The term ‘mouth guard’ as used herein refers to one specific implementation of a mouth-wearable sensing device and does not limit the scope of the disclosure to mouth guard form factors. The registration of the mouth-wearable sensing device 100 against the roof of the mouth functions to maintain a consistent and repeatable spatial relationship between sensors and the anatomical landmarks of the oral cavity (including the incisive papilla and the greater palatine vasculature), across wearing sessions and subject activities, such that physiological measurements can be reliably correlated with specific anatomical locations. In some implementations, the mouth-wearable sensing device 100 is formed as a mouthguard comprising a body shaped to fit over one or more teeth of the subject. In further implementations, the mouth-wearable sensing device 100 is formed as, or incorporated into, another intraoral appliance such as a dental retainer, a denture, an orthodontic appliance, or another structure suitable for sustained intraoral wear, etc.

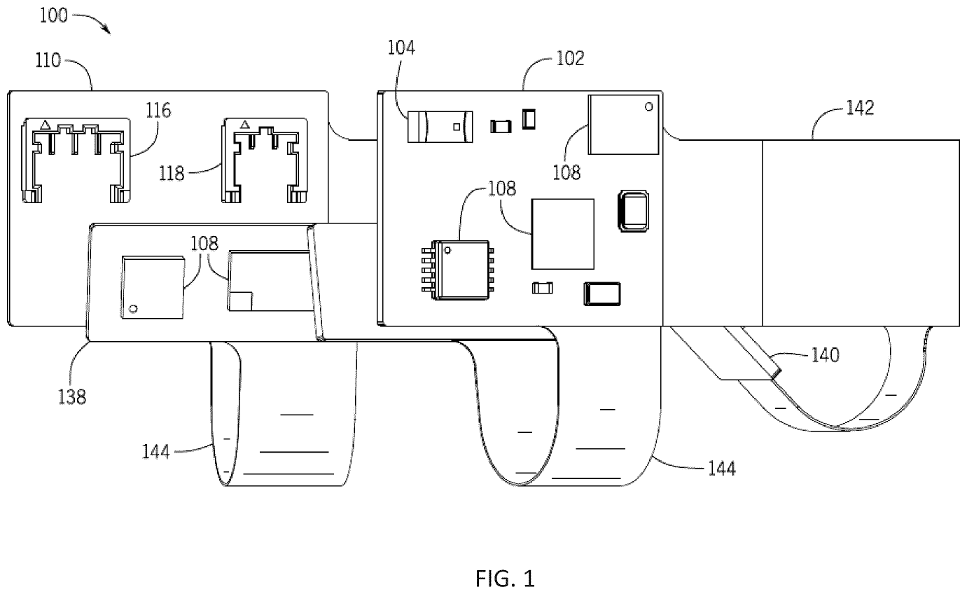

Referring now to FIG. 1, which shows a schematic plan view of an example mouth-wearable sensing device 100, in various implementations the mouth-wearable sensing device 100 includes a battery component 116 configured to provide operating power to the device, and a power source component 118 configured to distribute, condition, and/or manage power delivered to other device components. In some implementations, the battery component 116 and power source component 118 are located on power panel 110. In various implementations, the mouth-wearable sensing device 100 includes an antenna 104 and circuitry 108. In some implementations, circuitry 108 is located on exterior sensor panel 102. In some implementations, circuitry 108 is located on power panel 110. In some implementations, power panel 110 and interior sensor panel 138 are connected via inner loop 144. In some implementations, interior sensor panel 138 and exterior sensor panel 102 are also connected via inner loop 144. In some implementations, exterior sensor panel 102 and power panel 110 are connected via connector 142. In some embodiments, the battery component 116 is rechargeable and is configured to be charged via one or more charging modalities. In some embodiments, the mouth-wearable sensing device 100 is charged using an inductive (wireless) charging interface, in which the device is placed in or on a charging base station that transfers power via inductive coupling through the device body, eliminating the need for an exposed electrical port and supporting a fully sealed, waterproof device enclosure. In some embodiments, the device is charged via a waterproof electrical charging port—such as a USB-C port with an IP-rated waterproof seal or a proprietary sealed connector—that allows wired charging while maintaining protection against moisture ingress. In some embodiments, the device may support both inductive and wired charging modalities. In some embodiments, the charging base station is configured as a protective storage case that charges the device when stored therein.

In various implementations, circuitry 108 is configured to support operation of the mouth-wearable sensing device 100. In some implementations, circuitry 108 comprises a processor configured to coordinate operations of the mouth-wearable sensing device 100, including acquiring data from one or more sensors, processing or packaging that data, and controlling communication of the data to external devices. In some implementations, the processor of circuitry 108 comprises an ARM processor (e.g., an ARM Cortex-M series processor, or another low-power embedded processor suitable for intraoral wearable applications, etc.). In further implementations, circuitry 108 comprises a digital signal processor (DSP) configured to perform real-time signal processing on sensor data acquired from one or more sensors, including filtering, feature extraction, or other signal conditioning operations. In further implementations, circuitry 108 comprises one or more of: an analog-to-digital converter (ADC) to digitize analog signals from one or more sensors; a system on chip (SOC) integrating processing, memory, and communication functions; a power management integrated circuit (PMIC) to manage power delivery to components of the device; local memory or storage configured to buffer sensor data; and any other supporting electronics associated with the electrical architecture of the mouth-wearable sensing device 100, etc. In some implementations, circuitry 108 is configured to perform on-device edge processing of sensor data, which may comprise one or more of: signal filtering to remove noise or motion artifacts; event detection to identify force impact events or other physiologically significant occurrences; data compression or packaging to reduce transmission bandwidth; sensor data synthesis to fuse or combine data streams from two or more sensors into a combined output or derived physiological metric; and application of one or more trained machine learning models to generate severity metrics, classifications, or other outputs from sensor data, etc., all prior to or in lieu of transmission to an external device. In some implementations, circuitry 108 comprises a wireless transmitter configured to transmit sensor data to an external device. In some implementations, the external device comprises one or more of: a mobile computing device such as a smartphone or tablet; a dedicated base station or receiver; a wrist-worn computing device such as a smartwatch or wrist-worn health monitor; a workstation; or another suitable computing device, etc. In some implementations, circuitry 108 is configured to operate in a streaming mode in which sensor data is streamed in real time to a paired external device over a wireless communication protocol. In further implementations, circuitry 108 is configured to operate in an offload mode in which sensor data is stored locally in memory or storage of circuitry 108 during a period when a wireless connection to an external device is unavailable or not desired, and the stored sensor data is transmitted to the external device when a wireless connection is subsequently established. In some implementations, the mouth-wearable sensing device 100 is configured to switch between streaming mode and offload mode based on connectivity status, user configuration, activity type, or other operating conditions, etc.

In various implementations, the boards or sensor assemblies of the mouth-wearable sensing device 100 (e.g., exterior sensor panel 102, power panel 110, and interior sensor panels 138, etc.) communicate with one another wirelessly, without wired connectors between them. In some implementations, each board or sensor assembly of the mouth-wearable sensing device 100 comprises its own dedicated power source (e.g., an individual battery or energy storage element, etc.) configured to power the sensors and circuitry on that board or assembly independently of the other boards or assemblies. In some implementations, the dedicated power sources of the respective boards or sensor assemblies are each configured to be charged inductively, such that the mouth-wearable sensing device 100 can be charged without physical electrical connections between the boards or assemblies or to an external charging contact. In some implementations, the boards or sensor assemblies of the mouth-wearable sensing device 100 each comprise wireless communication circuitry configured to communicate sensor data and/or control signals to one or more of the other boards or assemblies over a short-range wireless communication protocol, forming a wireless personal area network (e.g., a PicoNet, etc.) embedded within the mouth-wearable sensing device 100. In further implementations, one board or assembly of the mouth-wearable sensing device 100 acts as a coordinator or central node of the wireless personal area network, receiving data from the other boards or assemblies and transmitting aggregated sensor data to an external device via the wireless transmitter of circuitry 108. This distributed wireless architecture eliminates wired interconnects between boards or assemblies, which may reduce mechanical stress at flex joints, simplify the physical structure of the device, and enable modular assembly and replacement of individual sensor assemblies, etc. In other implementations, the boards or assemblies of the mouth-wearable sensing device 100 are interconnected by a flexible wired bus (e.g., a flexible polyimide PCB bus sharing power, ground, and signal lines between rigid segments), and wireless inter-board communication is not employed.

In various implementations, the mouth-wearable sensing device 100 comprises one or more sensors configured to obtain physiological and kinematic data from the subject during wear. The sensors may include sensors of any suitable type and quantity, selected based on the physiological signals to be acquired. In some implementations, the sensors include one or more of: an optical sensor (e.g., a pulse oximeter 140 or photoplethysmography (PPG) sensor) to measure heart rate, peripheral oxygen saturation (SpO2), or other optically derived physiological signals; one or more thermistors 150 to measure body temperature; one or more force sensing resistors 146 to measure bite force; one or more inertial measurement units (IMUs) to measure linear and rotational acceleration; one or more accelerometers to measure linear acceleration; and one or more chemical sensors 148 to detect chemical constituents of fluids or gases present in the oral cavity, etc. In further implementations, the sensors include one or more of a salivary sensor, a humidity sensor, a carbon dioxide (CO2) sensor, and a chemical sensor array. These sensing modalities may be used individually or in combination to provide multi-modal physiological data. In some implementations, the intraoral placement of the pulse oximeter 140 proximate to the incisive papilla—which provides access to a well-perfused vascular bed proximate to the sphenopalatine vasculature—may provide an improved measurement site for SpO2 and blood flow characteristics in subjects for whom conventional peripheral measurement sites (e.g., fingertip or earlobe) may yield attenuated or unreliable signals due to reduced peripheral perfusion. Subjects with conditions associated with reduced peripheral blood flow (e.g., Raynaud's syndrome, peripheral vascular disease, hypothermia, or other conditions characterized by episodic or chronic reductions in peripheral perfusion, etc.) may benefit from this intraoral measurement site, which may be less susceptible to peripheral vasospasm or hypoperfusion in some subjects compared to extremity-based pulse oximetry, thereby potentially providing improved signal quality for SpO2 and heart rate measurement in such subjects.

In various embodiments, the optical sensor positioned proximate to the incisive papilla may be subject to intraoral-specific signal artifacts, including motion artifacts arising from jaw movement, optical interference from saliva pooling, pressure variation from occlusal loading, and ambient light leakage. To mitigate these artifacts, in some embodiments the mouth-wearable sensing device incorporates one or more of: mechanical isolation of the optical sensor within a recessed cavity or compliant enclosure formed in the device body to reduce pressure variation and motion coupling; optical shielding comprising an opaque barrier or light-blocking encapsulant to reduce ambient light ingress at the sensor aperture; pressure control features configured to maintain a target contact force between the optical sensor and the tissue of the incisive papilla; a signal quality index computed from one or more of the optical signal amplitude, signal-to-noise ratio, or motion sensor outputs, used to flag or discard low-quality optical readings; and adaptive filtering applied to the optical signal using motion data from one or more inertial measurement units to suppress motion-correlated noise components. In some embodiments, signal quality index thresholding is applied prior to computation of heart rate or SpO2 values to reduce the influence of artifact-contaminated segments on reported physiological values.

In various implementations, the mouth-wearable sensing device 100 supports a first sensor positioned proximate to the incisive papilla of the subject when the device 100 is worn, and a second sensor positioned proximate to the greater palatine vasculature of the subject when the device 100 is worn. The incisive papilla, (located at the anterior hard palate), provides proximity to the sphenopalatine vasculature, which supplies blood to the anterior hard palate and supports acquisition of optical physiological signals such as PPG and pulse oximetry. The greater palatine vasculature (located in the posterior hard palate near the maxillary molars) provides a vascular source suitable for temperature sensing. In some implementations, the first sensor is a pulse oximeter 140 positioned proximate to the incisive papilla, supported on exterior sensor panel 102. In some implementations, the second sensor is a first thermistor 150 positioned proximate to a first branch of the greater palatine vasculature, supported on a first interior sensor panel 138. In further implementations, the mouth-wearable sensing device 100 comprises a third sensor that is a second thermistor 150 positioned proximate to a second branch of the greater palatine vasculature, supported on a second interior sensor panel 138, providing bilateral temperature measurement and enabling cross-validation of temperature data.

In various implementations, one or more force sensing resistors 146 are supported by the structure of the mouth-wearable sensing device 100 and positioned within the molar region of the oral cavity to sense bite force of the subject when the device 100 is worn. In some implementations, a first force sensing resistor 146 is positioned in a first molar region and a second force sensing resistor 146 is positioned in a second molar region, providing bilateral bite force measurement. The molar regions correspond to locations of concentrated bite force within the oral cavity. The bilateral arrangement of force sensing resistors 146 in the first and second molar regions supports measurement of bite force on both the left and right sides of the oral cavity, enabling detection of asymmetric clenching patterns, postural imbalance, or differential responses to force impact events on each side of the jaw, etc. In some implementations, one or more force sensing resistors 146 are disposed on connectors (e.g., inner loop 144 or connector 142) that electrically interconnect boards or assemblies of the mouth-wearable sensing device 100, such that the connectors traverse the molar regions when the device 100 is worn and the force sensing resistors 146 register occlusal loading at those locations. In further implementations, the bite force data obtained from force sensing resistors 146 is provided to circuitry 108 for use in assessing postural control, jaw clenching responses associated with force impact events, or other physiological or biomechanical parameters, etc.

In some implementations, the locations of concentrated bite force in the first and second molar regions vary among individual subjects depending on each subject's occlusal anatomy, dentition, and jaw mechanics. Accordingly, in some implementations, the placement positions of the force sensing resistors 146 are determined on a subject-specific basis using intraoral characterization data representing the subject's oral anatomy (e.g., occlusal analysis data, bite force measurement data, or three-dimensional digital model data, etc.) to identify the locations within the oral cavity where bite force concentration is greatest for that individual subject. The force sensing resistors 146 are then positioned in the mouth-wearable sensing device 100 at those subject-specific locations, such that the force sensing resistors 146 are optimally aligned with the subject's actual bite force concentration points when the device 100 is worn.

In various implementations, the force sensing resistors 146 are configured to be maintained in a flat, substantially unloaded baseline state when the mouth-wearable sensing device 100 is not subjected to occlusal loading. In some implementations, the body or encapsulating structure of the mouth-wearable sensing device 100 (e.g., the biocompatible material forming the mouthguard body, etc.) is configured to support the force sensing resistors 146 in this flat baseline state, such that the encapsulating material does not pre-load or deflect the force sensing resistors 146 in the absence of applied bite force. This ensures that the force sensing resistors 146 register at or near a known baseline resistance value when no bite force is applied, and that deviations from that baseline accurately reflect applied occlusal loading. In some implementations, the encapsulating material is selected and configured to have a compliance sufficient to transmit bite force loads to the force sensing resistors 146 while maintaining the flat baseline state of the sensors in the absence of applied force.

In various implementations, the mouth-wearable sensing device 100 comprises one or more inertial measurement units (IMUs) and one or more accelerometers supported by the structure and positioned within the oral cavity to measure head kinetics when the device 100 is worn. In some implementations, a first IMU is mounted on a first sensor assembly (e.g., a first interior sensor panel 138) positioned proximate to a first branch of the greater palatine vasculature when the device 100 is worn, and a second IMU is mounted on a second sensor assembly (e.g., a second interior sensor panel 138) positioned proximate to a second branch of the greater palatine vasculature when the device 100 is worn. In some implementations, positioning the IMUs proximate to the first and second branches of the greater palatine vasculature co-locates the inertial sensors with thermistors 150 on the respective sensor assemblies, enabling a compact bilateral sensor arrangement on each side of the palate that measures both temperature and head kinetics from the same anatomical location. The posterior hard palate region proximate to the greater palatine vasculature corresponds to a position that is symmetrically disposed near the midsagittal plane of the oral cavity and proximate to the cranial base, which supports accurate measurement of both linear and rotational head acceleration. In further implementations, a first accelerometer is mounted on the first sensor assembly and a second accelerometer is mounted on the second sensor assembly. In some implementations, the first sensor assembly and the second sensor assembly each comprise a sensor panel (e.g., interior sensor panel 138) that supports both an IMU and a thermistor 150, enabling co-located inertial and temperature sensing at each of the first and second branches of the greater palatine vasculature. The intraoral position of the mouth-wearable sensing device 100 provides a rigid coupling to the skull via the dental arch, which may reduce signal attenuation and sensor decoupling associated with externally mounted sensors, such as those attached to helmets or skin.

In various implementations, sensor data obtained from force sensing resistors 146 and one or more other sensors of the mouth-wearable sensing device 100 is processed to assess postural control of the subject. In some implementations, changes in head and body posture of the subject result in changes in dental occlusion (e.g., the contact relationship between the upper and lower teeth, etc.), which produce measurable changes in the data obtained by force sensing resistors 146 positioned in the molar regions of the oral cavity, enabling assessment of postural state and postural changes over time from the intraoral sensor data. In further implementations, postural control metrics are derived from a combination of force sensing resistor data, IMU data, and accelerometer data, enabling characterization of the subject's postural engagement and postural response before, during, and after a force impact event.

In some embodiments, a postural control metric is derived from the bite-force signals obtained by the force sensing resistors using a feature-extraction and mapping pipeline. In one example pipeline, raw bite-force signals from the left and right force sensing resistors are segmented into time windows (e.g., one-second epochs), and one or more features are extracted from each window, including: (a) bilateral asymmetry index, computed as the normalized difference between left and right bite-force magnitudes; (b) bite-force variance within the window, indicative of postural stability; (c) tremor frequency, estimated by applying a frequency-domain transform (e.g., fast Fourier transform) to the bite-force time series to identify dominant oscillatory components; and (d) mean bite force. The extracted features are mapped to one or more postural state classifications or postural control metrics using a trained classifier or regression model. In some embodiments, the postural control metric comprises one or more of: an asymmetry index value, a variance metric, a tremor frequency estimate, or a composite postural stability score. In some embodiments, a calibration routine is performed prior to activity to establish subject-specific baseline values. During calibration, the subject performs one or more standardized tasks—such as quiet bilateral stance with eyes open, eyes-closed stance, and a defined perturbation task—and the bite-force features obtained during these tasks are used to define a subject-specific baseline distribution against which subsequent measurements are compared. Postural changes are then assessed by comparing real-time feature values against the calibrated baseline, with deviations beyond a threshold flagging a change in postural state.

In various implementations, a chemical sensor 148 is supported by the structure of the mouth-wearable sensing device 100 and positioned within the oral cavity such that, when the device 100 is worn, the chemical sensor 148 is exposed to air exhaled through the mouth of the subject. This placement enables the chemical sensor 148 to detect one or more chemical constituents of exhaled breath, which may include, without limitation, volatile organic compounds, carbon dioxide (CO2), humidity, or other breath biomarkers, etc. In some implementations, chemical sensor 148 comprises a salivary sensor configured to detect one or more biomarkers present in saliva of the subject, such as cortisol, electrolytes, glucose, proteins, or other salivary constituents indicative of physiological or metabolic state, etc. In some implementations, chemical sensor 148 comprises a humidity sensor configured to detect the relative humidity of the oral environment, which may vary as a function of respiratory rate, hydration status, or other physiological parameters, etc. In some implementations, chemical sensor 148 comprises a CO2 sensor configured to detect the concentration of carbon dioxide in air exhaled through the mouth of the subject, which may be indicative of respiratory function, ventilatory rate, or metabolic state, etc. In some implementations, chemical sensor 148 comprises a chemical sensor array comprising a plurality of individual sensing elements each responsive to a different chemical constituent, enabling multi-analyte detection from a single sensor assembly. In further implementations, chemical sensor 148 is disposed on a connector (e.g., inner loop 144 or connector 142) that extends from a sensor assembly (e.g., exterior sensor panel 102, which supports pulse oximeter 140) to other portions of the mouth-wearable sensing device 100, such that the connector traverses an airflow path within the oral cavity during exhalation. Analog signals from chemical sensor 148 may be digitized by an analog-to-digital converter of circuitry 108 and processed or transmitted by circuitry 108.

In some implementations, one or more chemical sensors 148 are positioned in the anterior region of the oral cavity (e.g., on a connector or sensor assembly located beneath or proximate to the anterior hard palate, etc.) to detect chemical constituents of air exhaled through the mouth of the subject (e.g., CO2, volatile organic compounds, or other breath biomarkers, etc.). In some implementations, one or more chemical sensors 148 are positioned in the posterior region of the oral cavity (e.g., on a connector or sensor assembly located proximate to the posterior hard palate or the molar regions, etc.) to detect chemical constituents present in saliva or in the posterior oral environment (e.g., salivary biomarkers, humidity, or other chemical analytes, etc.). In some implementations, a salivary sensor or humidity sensor is positioned in the posterior region of the oral cavity to take advantage of increased salivary pooling and exposure to posterior oral fluids. In some implementations, a chemical sensor array is positioned in the anterior region of the oral cavity, the posterior region of the oral cavity, or both, depending on the target analytes and the oral anatomy of the subject.

In various implementations, the mouth-wearable sensing device 100 is configured with a sensor complement selected for a particular target population or use case. In some implementations, the mouth-wearable sensing device 100 is configured for concussion detection and head impact monitoring, comprising one or more IMUs, one or more accelerometers, one or more force sensing resistors 146, and a pulse oximeter 140, enabling simultaneous measurement of head kinetics, bite force, and optical physiological signals during and after force impact events. In some implementations, the mouth-wearable sensing device 100 is configured for continuous patient monitoring in a clinical or non-clinical setting, comprising one or more thermistors 150 and one or more chemical sensors 148 (e.g., a CO2 sensor, a humidity sensor, or a salivary sensor), without IMUs or accelerometers, enabling monitoring of body temperature, respiratory function, and breath or salivary biomarkers in populations for whom inertial sensing is not required (e.g., hospital patients, older adults, or patients undergoing recovery monitoring, etc.). In some implementations, the mouth-wearable sensing device 100 is configured for sleep monitoring, comprising sensors selected to acquire physiological data characteristic of sleep states, such as thermistors 150 to monitor body temperature variation across sleep cycles, chemical sensors 148 to detect respiratory rate or CO2 concentration, and a pulse oximeter 140 to monitor SpO2 and heart rate, without force sensing resistors or inertial sensors that are unnecessary during sleep. In further implementations, the mouth-wearable sensing device 100 is incorporated into custom dentures or other full-arch dental appliances, enabling long-term physiological monitoring in populations who wear dental prostheses (e.g., older adults, long-term care patients, or others for whom a standalone mouthguard form factor is unsuitable, etc.).

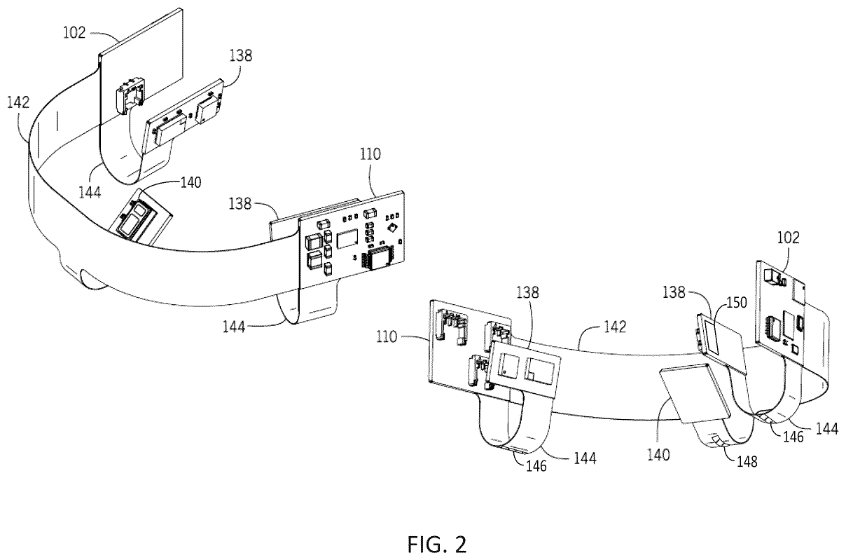

FIG. 2 shows a perspective view of an example mouthguard body of the mouth-wearable sensing device. In some implementations, inner loop 144 and connector 142 comprise a six-layer high-density interconnect (HDI) rigid-flex printed circuit board.

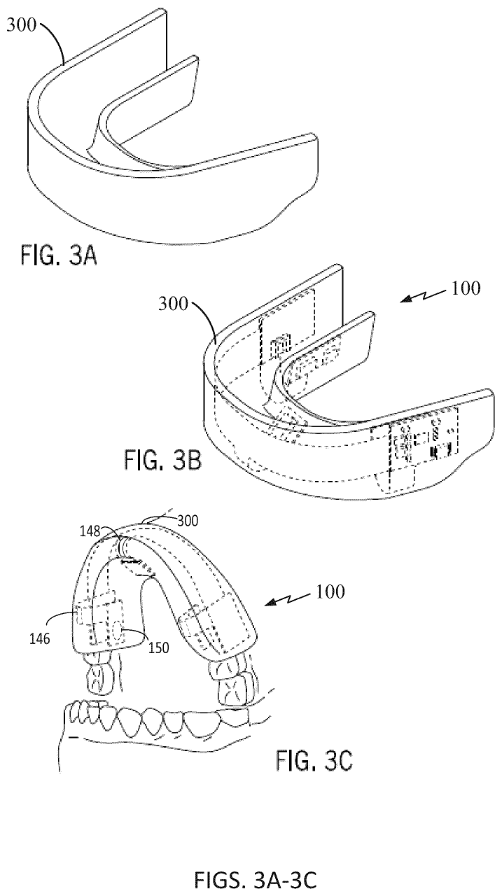

FIG. 3 is a perspective view illustrating an example mouth-wearable sensing device in a body formed of a biocompatible material. FIG. 3A depicts a body formed of a biocompatible material. In some implementations, the body is formed as a unitary or multi-layer structure that encapsulates and protects at least a portion of a mouth-wearable sensing device while remaining suitable for prolonged intraoral contact. In example implementations, the body may comprise polymethyl methacrylate (PMMA), (e.g., as a biocompatible encapsulation material for hardware within the mouthguard system). In other implementations, the body may comprise a thermoplastic polymer commonly used for mouthguards (e.g., ethylene-vinyl acetate (EVA) for boil-and-bite or custom-fit mouthguards, and/or other thermoformable polymers such as thermoplastic polyurethane (TPU) or PETG (polyethylene terephthalate glycol-modified), each selected based on a desired balance of comfort, durability, and impact absorption). In further implementations, the body (or one or more encapsulating layers thereof) may comprise a resilient elastomer such as medical-grade silicone, (e.g., to provide sealing, cushioning, and/or mechanical isolation) of embedded components while maintaining fit and intraoral comfort. In still other implementations, the mouthguard body may include one or more rigid or semi-rigid polymer layers (e.g., acrylic resin or a dual-layer composite including a softer inner layer and firmer outer layer) to provide structural reinforcement and/or abrasion resistance while preserving energy absorption and retention on the teeth. FIG. 3B depicts mouth-wearable sensing device 100 encapsulated in the body. FIG. 3C depicts mouth-wearable sensing device 100 encapsulated in the body shaped to fit over at least a portion of one or more teeth of the subject.

In various embodiments, the sensors are mechanically coupled to the body of the mouth-wearable sensing device 100 to maintain stable contact with the relevant intraoral tissue during wear. In some embodiments, the optical sensor positioned proximate to the incisive papilla is embedded within a recessed cavity formed in the palatal surface of the device body, such that the sensor aperture is positioned at a defined standoff distance from the tissue and contact pressure is maintained by the fit of the device body against the palate. In some embodiments, a compliant layer—such as a medical-grade silicone pad or elastomeric cushion—is disposed between the sensor and the tissue-contacting surface of the device body to distribute contact pressure, reduce focal pressure points, and accommodate minor positional variation during use while maintaining optical coupling. In some embodiments, the device body incorporates mechanical retention features—such as a dental arch fit, occlusal stops, or palatal registration surfaces—that resist displacement of the device during jaw movement or impact events, thereby maintaining consistent sensor-to-tissue positioning. In some embodiments, the thermistors and inertial measurement units positioned at the posterior palatal regions are similarly embedded within or secured to the rigid sections of the rigid-flex circuit board substrate and encapsulated within the biocompatible body material, providing structural support while maintaining thermal and mechanical contact with the adjacent tissue.

In some implementations, the mouth-wearable sensing device 100 is formed as part of, an oral appliance comprising at least a dental retainer or a denture.

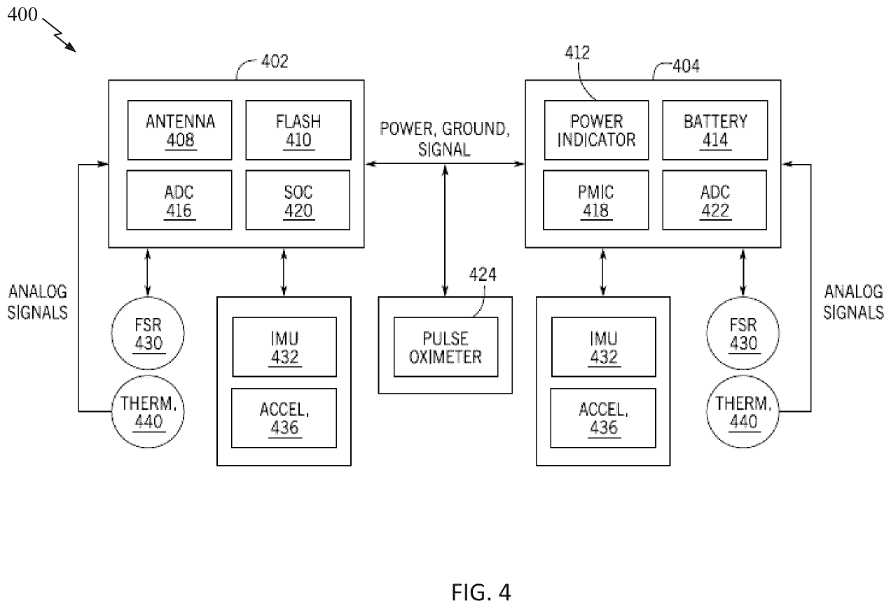

FIG. 4 is block diagram 400 illustrating example electrical architecture of the mouth-wearable sensing device, including example sensors and supporting electronics. All components described in block diagram 400 may be integrated into the depictions of mouth-wearable sensing device 100 as presented in FIG. 1, FIG. 2 or FIG. 3. For instance, in some implementations antenna 408 presented in FIG. 4 corresponds with the antenna 104 as presented in FIG. 1. In other instances, the pulse oximeter 424 corresponds with the pulse oximeter 140 as presented in FIG. 1. In other implementations, a first portion 402 corresponds with the exterior sensor panel 102. In some implementations, a second portion 404 corresponds with a power panel 110. In some implementations, inertial measurement units (IMU) 432 and accelerometer 436 are located on interior sensor panel 138.

In some implementations, the mouth-worn wearable sensing device comprises a first portion 402, which comprises an antenna 408, flash 410, analog to digital converter (ADC) 416, and system on chip (SOC) 420. In some implementations, antenna 408 comprises a 2.4 GHz chip antenna. In some implementations, the 410 flash comprises a 128 MB SPI NOR Flash module. In some implementations, the SOC 420 comprises an ultra low-power ARM Cortex-M4 system on chip. In some implementations, ADC 416 comprises a TI ADS1115 Analog to Digital Converter.

In some implementations, the mouth-wearable sensing devices comprises a second portion 404, which comprises a power indicator 412, a battery 414, analog to digital converter (ADC) 422, and power management integrated circuit (PMIC) 418. In some implementations, ADC 422 comprises a TI ADS1115 Analog to Digital Converter. In some implementations, the PMIC 418 is Nordic nPM1300 Power management integrated circuit.

In some implementations, the mouth-wearable sensing devices comprises a plurality of sensors. In some implementations, the plurality of sensors includes one or more of: a pulse oximeter 424 to measure and/or monitor heart rate and peripheral capillary oxygen saturation (SpO2); an inertial measurement unit 432 to measure and/or monitor linear and rotational acceleration; an accelerometer 436 to measure and/or monitor linear acceleration; a force sensing resistor 430 to measure and/or monitor bite force; a thermistor 440 to measure and/or monitor body temperature of a subject; etc.

In various implementations, the mouth-wearable sensing device 100 comprises one or more output components configured to provide feedback to the wearer or to observers. In some implementations, the mouth-wearable sensing device 100 comprises an indicator (e.g., a light emitting diode (LED) or other visual output element, etc.) positioned on the device and visible when the device is worn or removed from the mouth, configured to display a status indication based on sensor data or on the output of circuitry 108. In some implementations, the indicator is configured to display a color-coded status corresponding to a detected or estimated concussion risk level (e.g., displaying a first color such as green indicating no detected risk, displaying a second color such as yellow indicating a detected impact or physiological change that may warrant medical evaluation, and displaying a third color such as red indicating a detected impact or physiological response associated with a likely concussive event, etc.). In further implementations, the color-coded status is determined based on a threshold applied to a severity metric output by circuitry 108 or by an external device. In still further implementations, the thresholds associated with the color-coded status levels are programmable, user-configurable, adaptively determined by a machine learning model, or pre-programmed based on established clinical or regulatory standards, etc.

In some implementations, the mouth-wearable sensing device 100 comprises a haptic output element (e.g., a vibration motor or piezoelectric actuator, etc.) configured to provide tactile feedback to the wearer. In some implementations, the haptic output element is configured to generate a haptic signal when sensor data obtained by the mouth-wearable sensing device 100 meets or exceeds a threshold indicative of approaching a force level associated with concussive injury risk, enabling the wearer to perceive the feedback during activity and modify their behavior accordingly (e.g., enabling an athlete to adjust tackling technique or body positioning in response to cumulative impact feedback delivered in real time during play, etc.). In some implementations, the thresholds triggering the haptic output are programmable, adaptively determined, or pre-programmed, and may correspond to cumulative force exposure thresholds, single-impact severity thresholds, or other clinically or operationally relevant criteria, etc.

In various implementations, the combination of the anatomically targeted intraoral sensor placement of the mouth-wearable sensing device 100 with machine learning processing of the resulting multi-modal sensor data produces a technical improvement in physiological monitoring that could not be achieved by generic sensor placement or conventional signal analysis alone. In some implementations, the intraoral placement of sensors proximate to the incisive papilla and the greater palatine vasculature provides access to vascular signals that may not be readily obtainable from externally mounted sensors, and the concurrent measurement of bite force, head kinetics, temperature, and optical physiological signals from these anatomically specific locations produces a fused, time-aligned multi-modal data stream not previously available for TBI assessment. In some implementations, the machine learning models process this multi-modal intraoral sensor data stream to detect patterns and correlations across sensor modalities (e.g., correlations between bite force magnitude, rotational acceleration, and photoplethysmographic signal perturbation, etc.) that are specific to force impact events of a type and severity associated with concussive injury, and that are not detectable from any single sensor modality or from externally mounted sensors. The output of the machine learning model (e.g., a severity metric, classification, or behavioral recommendation, etc.) represents a specific, technically grounded determination based on the particular sensor data architecture of the mouth-wearable sensing device 100, and is distinct from a general-purpose or abstract mathematical operation.

In various implementations, circuitry 108 and/or an external device is configured to process sensor data obtained by the mouth-wearable sensing device 100 and to generate one or more data outputs formatted to satisfy the data reporting requirements of applicable sports safety standards or regulatory bodies. In some implementations, the data output is formatted to satisfy the requirements of World Rugby or another sporting body that mandates performance or sensing criteria for instrumented mouthguards used in competitive sport, etc. In further implementations, the data output includes one or more of: peak linear acceleration; peak rotational acceleration; bite force measurements; heart rate; SpO2; body temperature; and event timestamps, in a format compatible with the reporting requirements of the applicable standard.

In various implementations, a plurality of mouth-wearable sensing devices 100 are deployed and operated simultaneously, each worn by a respective subject, and each communicatively coupled to a shared base station, receiver, or computing device that receives sensor data from each of the plurality of devices. In some implementations, the shared base station is configured to aggregate and display physiological data from each of the plurality of mouth-wearable sensing devices 100, enabling real-time group-level monitoring of all subjects in the deployment. In some implementations, the plurality of mouth-wearable sensing devices 100 are deployed in a sports context (e.g., a football team, a rugby team, or another contact sport team, etc.) enabling monitoring of an entire team simultaneously by coaches, athletic trainers, or medical personnel. In further implementations, the plurality of mouth-wearable sensing devices 100 are deployed in a military context (e.g., a military unit engaged in training or operational activities, etc.) enabling monitoring of each member of the unit by command or medical personnel. In still further implementations, the plurality of mouth-wearable sensing devices 100 are deployed in a clinical or institutional context (e.g., a hospital ward, a long-term care facility, or a rehabilitation center, etc.) enabling concurrent patient monitoring by clinical personnel. In some implementations, the base station communicates with each of the plurality of mouth-wearable sensing devices 100 over a short-range wireless protocol, and aggregates the received data for transmission to one or more networked computing devices for storage, analysis, and reporting.

In some implementations, the present disclosures provides a battery-powered mouth-wearable sensing device that integrates inertial measurement units (IMU), thermistors, force sensitive resistors, and an infrared optical sensor on a high-density rigid-flex printed circuit board (PCB), strategically positioned within the oral cavity for anatomically targeted signal acquisition. In some implementations, physiological data is wirelessly transmitted over Bluetooth Low Energy (BLE) to a companion mobile application that provides intuitive and real time visualization and analytics.

In one implementation, the system integrates multimodal physiological sensing into a compact, intraoral wearable device using a custom rigid-flex PCB form factor for optimal sensor placement. The architecture includes six primary sensing modalities, a central processing unit with Bluetooth Low Energy (BLE) connectivity, a highly integrated power management unit, and local Flash data storage. In some implementations, the device communicates with a mobile application for data processing, visualization, and algorithmic detection of TBI. FIG. 2 shows a detailed block diagram of the system.

In one example, sensor selection was based on ability to capture high-fidelity physiological signals relevant to TBI, namely head kinetics, bite force (e.g., related to postural control), temperature, and heart rate (e.g., autonomic functions). Similarly, assessment of autonomic and postural control requires careful consideration regarding sensor placement. The intraoral location of the device is ideal, as the mouth maintains direct proximity of the mandible and maxilla for assessing bite force and postural engagement, contains necessary vascular structures for accurate body temperature and photoplethysmography (PPG) measurements, and provides a more rigid coupling to the center of mass of the skull for the collection of intracranial measures of head impact. Table I shows the list of selected sensors with their respective location within the mouth, and associated biomarker.

TABLE I
Summary of Sensing Components and Physiological Targets

| Sensor | Part Number | Range | Biomarker |
|---|---|---|---|
| Force Sensing | A201-100 | 0 to 100 lb (445N) | Bite Force |
| Resistor | | | |
| Thermistor | MA300TA | 0 to 50° C. | Body Temperature |
| Pulse oximeter | MAX30102 | HR: 30 to 250 bpm, | Heart Rate, SpO₂ |
| | | SpO₂: 70 to 100% | |
| Accelerometer | ADXL375 | ±200 g | Linear Acceleration |
| Inertial | ICM-20948 | ±2 to ±16 g (accel), | Linear & Rotational |
| Measurement | | ±250 to ±2000 dps | Acceleration |
| Unit | | (gyro) | |

For the assessment of postural control, one implementation considers the functional relationship that exists between the sternocleidomastoid and the masseter. As both of these muscles are part of the stomatognathic system (i.e., the complex network of structures including the temporomandibular joint (TMJ), teeth, jaws, and associated muscles, as related to chewing, speaking, and other oral functions), the deployment of force sensing resistors (FSRs) was included to assess an individual's bite force. These sensors were placed in the molar region of the mouth, specifically at the second molar, given the ability to produce the maximum bite force.

For the assessment of autonomic features (e.g., heart rate, blood oxygen saturation (SpO2), and body temperature), several sensor deployments are included in this device design, such as PPG and thermistors.

PPG sensors are usually placed in locations that require minimum transmission intensity and blood vessels are close to the surface. Given the anatomy of the mouth, proximity of the incisive papilla was chosen given its prominence as a significant anatomical landmark. Here, the sphenopalatine vasculature provides a well-perfused vascular bed at the anterior hard palate, supporting viable PPG deployment.

Thermistors are also involved in the design of this system for the collection of accurate body temperature data. Although oral body temperature is commonly taken, this is usually done sublingually (i.e., under the tongue) due to increased vascularity. However, due to the development of a novel, self-contained mouthguard system, proximity to a new vascular source is necessary. To reduce the burden in the anterior palate, these thermistors were placed near the greater palatine vasculature, located in the posterior hard palate, typically near the maxillary molars.

Accurate measurement of head kinetics requires careful consideration of both placement and sensor dynamic range. Thus, the system integrates a total of four sensors: two Inertial Measurement Units (IMU) with low-range accelerometer, gyroscope, and magnetometer, and two high-range discrete accelerometers to achieve wide dynamic coverage of head motion.

In some implementations, the sensors are placed on the left and right inner surface of the device, symmetrically near the midsagittal plane and as close as possible to the center of the cranial base and cervical spine. In some examples, this configuration (1) reduces the effects of rotational artifacts and moment arms that can occur if the sensors are located further from the rotation axis, and (2) yields a more accurate measurement of rotational acceleration which is the predominant predictor of injury severity in TBI studies. The intraoral location provides rigid coupling with the skull via the maxillary dental arch. In some examples, this reduces damping and sensor decoupling errors often seen in helmet or skin mounted systems. In some implementations, the bilateral redundancy of both units allows for cross validation of motion data.

Processing and Power: In some implementations, the device is designed as a fully wireless, battery powered intraoral sensing platform. In an example implementation, the central processing unit comprises the Nordic nRF52840, an ultra-low-power ARM Cortex-M4 System on Chip (SoC) with integrated BLE 5.0, multiple I2C and SPI buses, and a built-in 12-bit ADC. The highly multiplexed GPIO allow for more flexible routing of peripheral traces given the size constraints. Power management is handled by the Nordic nPM1300 Power Management Integrated Circuit (PMIC), which is designed to work tightly with the nRF52840. The nPM1300 is a highly integrated PMIC that includes dual buck and low-dropout voltage regulators, power path management (e.g., automatic detection of power source), and power protection features. The battery temperature is monitored via a Negative Temperature Coefficient (NTC) thermistor, which triggers a system shutdown upon thermal irregularities.

In some implementations, FSR and thermistor are digitized using the TI ADS1115 Analog to Digital Converter (ADC). This ADC has a 16-bit resolution and a programmable sampling rate of up to 860 Hz. It also offers a programmable gain amplifier (PGA) with full-scale ranges from ±0.256 V to ±6.144 V, ideal for capturing subtle variations in both intraoral temperature and force exertion of bite events.

On-board Storage: Data storage needs vary depending on the type of activity and the limitations of the technology. For example, in use cases where real-time wireless connectivity to an external device is unavailable or intermittent—such as in certain field sports or outdoor activities—the device stores player data locally, which is then offloaded to a central device (e.g., phone, computer) when in range. As such, in some implementations, the device is equipped with 128 MB of SPI NOR Flash to store timestamped data during long, away sessions. Based on the sample rate of the sensors, frequency of collection, and size of each measurement, the selected storage size is calculated to be adequate to store a substantial duration of high-fidelity, timestamped sensor data during extended away sessions. In use cases where the device remains within continuous wireless range of a connected external device, the system can offload live data to the centralized device over BLE.

In some implementations, the design is implemented on a six-layer high-density interconnect (HDI) rigid-flex PCB that conforms to the anatomical structure of the upper oral cavity to support accurate placement of sensors based on their physiological access points. In an example implementation, the layout consists of two outer and three inner rigid FR4 sections. Two lateral rigid substrates are located on the left and right posterior portions of the device, and one central section under the upper incisors. Connected to the posterior sections are the last two rigid sections, hosting the IMU and high-g accelerometers. A flexible polyimide bus connects all of the rigid segments, sharing power, ground, and peripheral signals. The PCB has a custom stack up that consists of six layers with dedicated power and ground planes to minimize electro-magnetic interference (EMI) and noise coupling in the analog traces. The two inner layers carry signal and a ground plane through the flexible substrate to the rigid sections. Due to form factor constraints, the board incorporates micro vias and buried vias to accommodate for the dense routing of fine-pitch ball grid array (BGA) components such as the nRF52840 and nPM1300. The RF section consists of a 2.4 GHz chip antenna and a single-ended (SE) 50Ω impedance matching network. In this example implementation, a flexible polyimide bus connects the rigid segments, sharing power, ground, and peripheral signals via wired interconnects; in alternative implementations, inter-board communication may instead be implemented wirelessly, as described herein with respect to a distributed wireless architecture.

In some implementations, the firmware is built on the Zephyr Real-Time Operating System (RTOS) due to its extended support for wireless de-vices and built-in device drivers. Zephyr handles BLE pairing and communication, sensor data acquisition, PMIC control, task scheduling, and status indication with a built-in LED. Sensor communication is setup with interrupt-driven Direct Memory Access (DMA) channels. Each redundant sensor pair has access to a single I2C or SPI bus. For example, the two right IMU and high-g accelerometer are connected to the SPI1 channel, while the opposite is connected to SPI2. This setup ensures minimized latency and throughput optimization by reducing discrete CPU instructions.

In long distance activities where the device cannot send live updates to the server, the firmware implements on-board logging using a 128 MB SPI NOR Flash module. Each sensor reading is timestamped and stored in a SRAM buffer. Once the buffer is filled, data is written to flash using a Zephyr workqueue thread. This full configuration ensures 1) no blocking with the main execution thread and 2) no executive writing to the flash is done. Along with the sensor readings, metadata such as synchronization markers and start time is stored for longer sessions where the older data in flash may be overwritten. Once the device reestablishes BLE connection, it enters a data offload mode where the stored records are restored in chronological order. Due to the lack of a real-time clock (RTC), (given space constraints) timing synchronization occurs with respect to the boot time of the device, and adjustments to Unix time are handled by the mobile companion app.

In some implementations, Bluetooth communication and pairing is organized around custom Generic Attribute Profile (GATT) services and characteristics. While standard GATT profiles exist for Heart Rate and Health Thermometer, custom profiles were necessary due to the lack of a RTC and need for data synchronization. Instead, timestamps are retrieved from a system-wide timer initialized at boot time. Each GATT service encapsulates characteristics related to the physiological value being measured and their update rate. There are four services: Heart Rate, Health Thermometer, Motion, and Bite Force, each containing relevant sensor data and timestamp. For example, the Bite Force service has two 16-bit bite force variables (left and right bite) and a 32-bit timestamp encompassed in a struct. Decoding of the custom services is handled by the mobile companion app, updating the UI as needed.

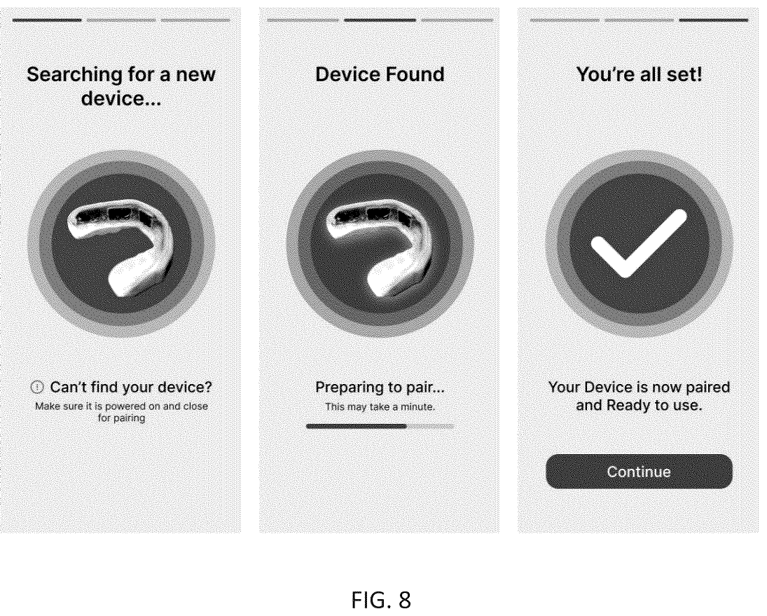

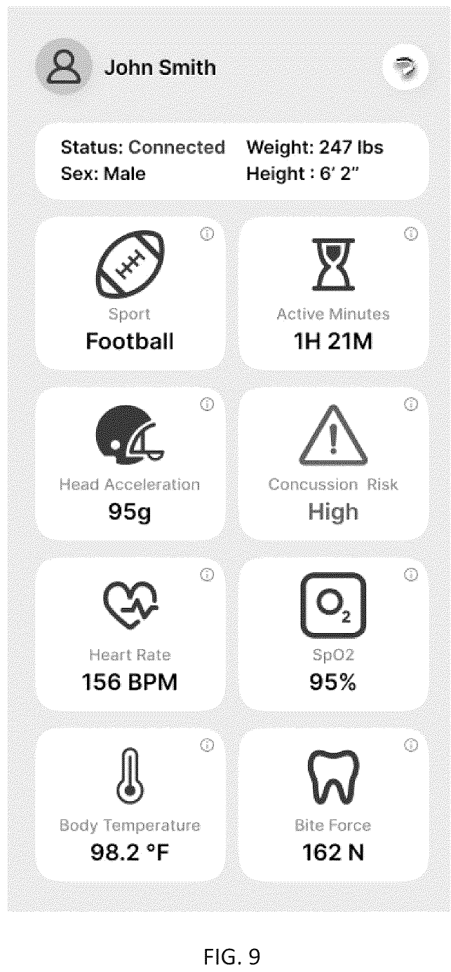

In one example, as the system is designed around physiologic monitoring of athletes it is necessary to present information on a paired companion via a simple and intuitive user interface. FIG. 8 and FIG. 9 depict a simplistic flow of the pairing process and a sample dashboard setup for this companion app. In pairing, a stepwise process is presented to the user that includes troubleshooting steps and visual feedback for process stages (FIG. 8). Following the pairing process, the user is directed to a live dashboard (FIG. 9) that presents information related to all biomarkers denoted in Table I (e.g., bite force, heart rate, SpO2, body temperature, and head kinetic data). In addition, this dashboard presents information on active minutes related to a specific athletic activity.

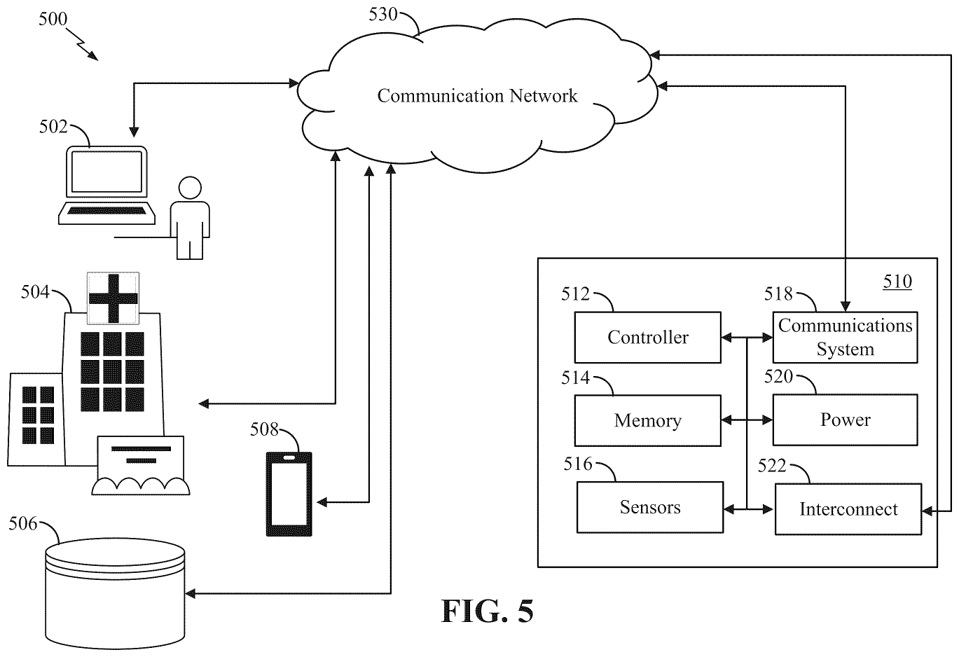

Referring now to FIG. 5, a block diagram is shown conceptually illustrating an example system 500 in which the mouth-wearable sensing device may operate, including transmission of physiological data over a communication network for storage, processing, and/or display.

In some implementations, the system 500 includes one or more external computing devices, such as a workstation or computing device 502, a facility system 504 (e.g., a clinic, training facility, hospital system, or other authorized monitoring station), a database or data repository 506, and/or a mobile computing device 508 (e.g., a smartphone executing a companion application). These external computing devices may be communicatively coupled via a communication network 530, which may include one or more wired and/or wireless networks (e.g., the Internet, a local network, a cellular network, a WiFi network, a Bluetooth connection, or combinations thereof).

In various implementations, the mouth-wearable sensing device includes, or is associated with, a device subsystem 510 (e.g., electronics integrated with the mouth-wearable) that comprises one or more functional components. For example, subsystem 510 may include a controller 512 configured to coordinate operations of the device, a memory 514 configured to store instructions and/or data, and a set of sensors 516 configured to obtain physiological and/or kinematic data from a subject. In some implementations, the set of sensors 516 includes the sensors described in FIG. 4.

In some implementations, the subsystem 510 further includes a power component 520 configured to supply and/or manage power for the controller 512, memory 514, sensors 516, and communications system 518. In certain implementations, the subsystem 510 also includes an interconnect 522 configured to couple internal components and/or to route power, ground, and signal pathways between components (including between physically separated regions of a rigid-flex circuit board).

In some implementations, the sensors 516 include a plurality of sensing modalities configured to obtain multi-modal data, such as inertial sensing, force sensing, optical sensing, and temperature sensing, as described in the FIG. 4, and the controller 512 may package and time-associate such sensor data for transmission and/or storage. In certain implementations, when a network connection is unavailable or intermittent, the controller 512 may store sensor data locally in memory 514 (e.g., as timestamped records) and later offload the stored data to the mobile computing device 508 or another external system via the communications system 518 when connectivity is reestablished.

In various implementations, the external computing devices 502, 504, 506, and/or 508 may be configured to receive sensor data and to provide one or more outputs based on the received data, such as visualizations, alerts, reports, or stored records, depending on user permissions and deployment context. For example, in some implementations the mobile computing device 508 may decode transmitted sensor values and update a user interface in real time, while the database 506 may store historical data for longitudinal monitoring or subsequent analysis.

In some implementations, one or more of the computing device 502, facility system 504, data repository 506, and/or mobile device 508 may be configured to generate and provide reports, alerts, and/or visualizations to authorized personnel-including, by way of example, coaches, athletic trainers, medical providers, caregivers, and/or command personnel-regarding rapid physiological changes following a force impact event, thereby supporting decisions such as whether an athlete or service member should return-to-play or return-to-duty. In further implementations, the reports may be provided to caregivers or other authorized users to assist in determining whether additional medical evaluation or assistance may be warranted following a fall event, such as for an older adult.

In some implementations, as used herein, a “force impact event” comprises an occurrence in which an external mechanical force is applied to a subject (or to a portion of the subject's body) in a manner that produces a measurable change in at least one physiological or kinematic signal captured by the mouth-wearable sensing device. In example implementations, a force impact event may include a head impact event (e.g., an impact associated with concussion risk), a collision, a rapid acceleration/deceleration event, a blast-related event, a fall event, or a motor vehicle accident event, each of which may be associated with changes in inertial measurements and/or rapid physiological response signals. In some implementations, a force impact event may be identified or characterized based on inertial sensing data (e.g., accelerometer and/or gyroscope outputs) in combination with one or more additional sensor modalities (e.g., bite-force sensing, temperature sensing, and/or optical physiological sensing), including embodiments in which baseline physiological data is compared to event-associated physiological data to determine a magnitude or profile of response.

As used herein, “intraoral characterization data” refers to data representing intraoral structures, intraoral geometry, or intraoral function of a subject. Intraoral characterization data may include, without limitation, one or more of: three-dimensional geometric data of the oral cavity obtained from an intraoral scan, including digital impressions or three-dimensional surface models of teeth, gum line, palate, and adjacent intraoral tissues, acquired using an intraoral surface imaging system (e.g., a structured-light scanner, a laser scanner, or an intraoral camera system configured to capture overlapping images and/or surface information of intraoral anatomy to generate a three-dimensional surface representation, etc.); two-dimensional or three-dimensional imaging data of intraoral anatomy obtained using a radiographic imaging system (e.g., X-ray, cone beam computed tomography (CBCT), or periapical imaging, etc.), which may provide cross-sectional or volumetric anatomical data supplementing the three-dimensional surface representation; occlusal analysis data describing occlusal contacts, contact timing, contact area, force distribution, or bite force measured during jaw closure, obtained using an occlusal analysis system (e.g., a pressure-sensitive occlusal film system, a digital occlusal analysis device, or a T-Scan or equivalent occlusal force measurement system, etc.); bite registration data obtained using physical or digital bite registration techniques, including centric relation records, maximum intercuspation records, or protrusive and lateral records; dental cast or model data derived from physical or digital impressions of the dentition; and any other measurement or characterization of the subject's intraoral anatomy, dentition, or jaw function suitable for use in designing or customizing an intraoral appliance, etc. Intraoral characterization data may be obtained using one or more instruments or techniques, used individually or in combination, and may be processed to generate a three-dimensional digital model of the oral cavity suitable for device design and sensor placement determination.

Although FIG. 5 depicts a particular arrangement of elements, it should be appreciated that the illustrated system 500 is provided as an example, and that additional elements may be included or certain elements omitted. For example, one or more of the external computing devices 502, 504, 506, 508 may be combined, replicated, or replaced with other devices, and the communication network 530 may include one or more communication links appropriate to the particular implementation.

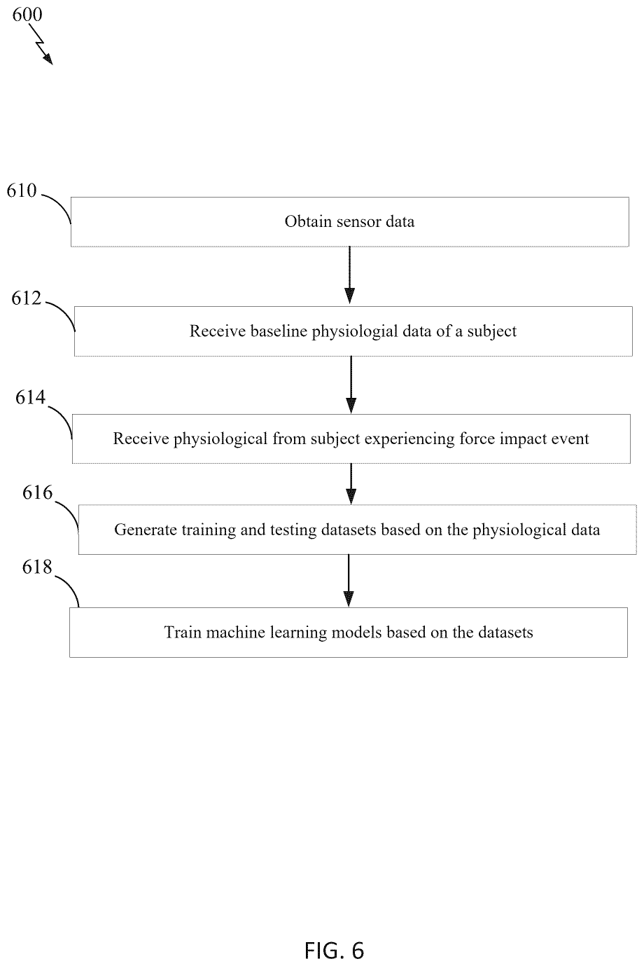

Referring now to FIG. 6, a flow diagram is shown for an example process 600 for preparing training data and training one or more machine learning models based on physiological sensor outputs obtained using the mouth-wearable sensing device. In some examples, a device subsystem 510 in connection with FIG. 5 can be used to perform example process 600. However, it should be appreciated that any suitable apparatus or means for carrying out the operations or features described herein may perform process 600.

In some implementations, process 600 may be implemented by one or more processors of the mouth-wearable sensing device (e.g., a system-on-chip), by one or more processors of an external computing device (e.g., a mobile device executing a companion application), or by a combination thereof. For example, sensor data may be obtained at the mouth-wearable sensing device and then transmitted (e.g., wirelessly) for storage, preprocessing, and model training at an external device, including in implementations that support on-device logging with subsequent offload.

At step 610, process 600 includes obtaining sensor data. In various implementations, the sensor data is obtained from a plurality of sensors of the mouth-wearable sensing device and may include inertial data (e.g., from an IMU and/or accelerometer), force data (e.g., bite-force signals), temperature data (e.g., from thermistors), and optical physiological data (e.g., photoplethysmography and/or pulse oximetry signals), in any suitable sampling configuration and/or sampling rates, and/or any data from sensors as described in FIG. 4.

At step 612, process 600 includes receiving baseline physiological data of a subject. In some implementations, the baseline physiological data is obtained during a period in which the subject is not experiencing a force impact event and may be collected to represent a baseline state for one or more sensor modalities. For example, baseline data may be collected to characterize typical signal ranges, trends, or distributions for a given subject prior to a force impact event.

At step 614, process 600 includes receiving physiological data from a subject experiencing a force impact event.

At step 616, process 600 includes generating training and testing datasets based on the physiological data. In some implementations, generating the datasets includes organizing and storing the baseline physiological data (block 612) and event-related physiological data (block 614) into datasets suitable for training and evaluation. For example, this may include time-aligning multi-sensor streams, segmenting the sensor data into one or more time windows (e.g., baseline windows and event windows), optionally filtering or normalizing the sensor data, and dividing the resulting records into training and testing sets. In implementations where data is logged locally, the datasets may be generated after data offload from local storage, including where each sensor reading is timestamped and stored for later processing.

At step 618, process 600 includes training one or more machine learning models based on the datasets. In some implementations, the one or more machine learning models include one or more time-series models configured to process the multi-sensor physiological data to generate an output indicative of a physiologic response or a concussion-like event. By way of example and not limitation, the one or more time-series models may include convolutional neural networks (CNNs) and/or recurrent neural networks (RNNs), including long short-term memory (LSTM) networks, to learn temporal dependencies and features from time-aligned sensor streams. In certain implementations, the models are trained using sensor-fusion inputs that combine two or more modalities (e.g., inertial signals, bite-force signals, temperature signals, and/or optical physiological signals) as fused feature streams to improve robustness and reduce false positives. In some implementations, at least a portion of the trained model(s) may be deployed for edge computation on the mouth-wearable sensing device (e.g., to support low-latency assessment), while training, refinement, or periodic updating may be performed on an external computing device using accumulated datasets over multiple sessions.

Although FIG. 6 depicts steps 610-618 in a particular order, it should be appreciated that one or more blocks may be omitted, combined, repeated, or performed in a different order depending on the implementation. For example, in certain implementations, baseline data (step 612) may be updated over multiple sessions, and the training/testing dataset generation of step 616 may be performed periodically as additional event force impact data becomes available.

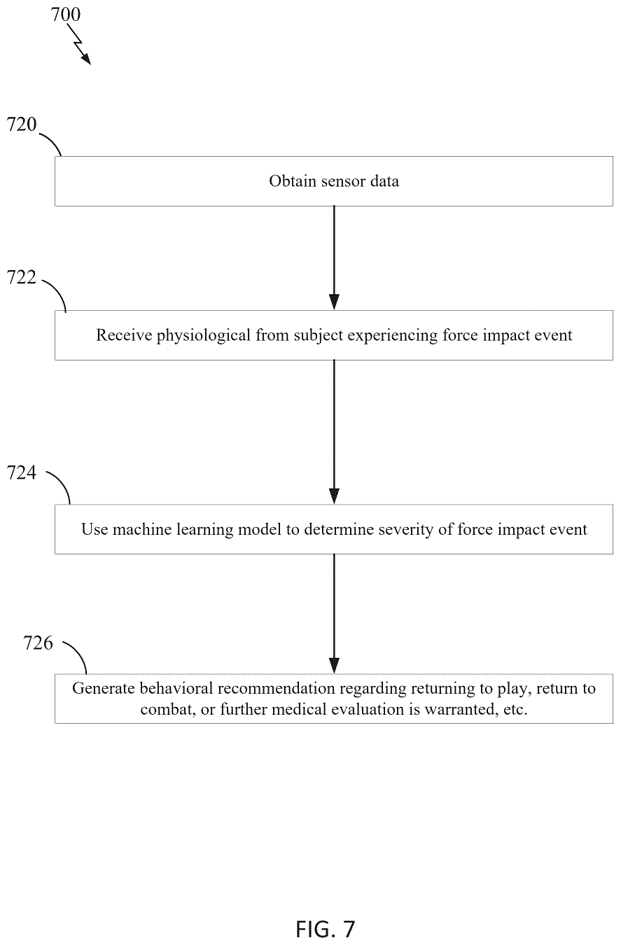

Referring now to FIG. 7, a flow diagram is shown for an example process 700 for generating an output indicative of a severity of a force impact event and generating a behavioral recommendation based thereon. In some examples, a device subsystem 510 in connection with FIG. 5 can be used to perform example process 700. However, it should be appreciated that any suitable apparatus or means for carrying out the operations or features described herein may perform process 700.

At step 720, process 700 includes obtaining sensor data. In various implementations, the sensor data is obtained from a plurality of sensors of the mouth-wearable sensing device and may include inertial data (e.g., IMU and/or accelerometer outputs), force data (e.g., bite-force signals), temperature data (e.g., thermistor outputs), and optical physiological data (e.g., photoplethysmography and/or pulse oximetry signals), in any suitable sampling configuration, and/or any data from sensors as described in FIG. 4.

At step 722, process 700 includes receiving physiological data from a subject experiencing a force impact event.

At step 724, process 700 includes using a machine learning model to determine severity of the force impact event. In some implementations, the machine learning model is configured to analyze one or more time-aligned sensor streams obtained at step 720 and/or physiological data received at step 722, and to output a severity metric, score, classification, or other quantitative or categorical indication representative of the force impact event.

At step 726, process 700 includes generating a behavioral recommendation regarding returning to play, return to combat, or whether further medical evaluation is warranted, based at least in part on the severity determined at step 724.

In some implementations, the behavioral recommendation is generated for presentation to authorized personnel, such as coaches, athletic trainers, medical providers, caregivers, and/or command personnel, to assist in decisions related to return-to-play or return-to-duty. In further implementations, the behavioral recommendation may include an indication that additional medical evaluation is recommended following a detected event (including a fall event in an older adult scenario), consistent with broader high-risk contexts described for the disclosed sensing platform.

Although FIG. 7 depicts steps 720-726 in a particular sequence, it should be appreciated that one or more blocks may be omitted, combined, repeated, or performed in a different order depending on the implementation. For example, in some implementations, at least a portion of the model of step 724 may be executed on-device to provide lower-latency outputs, while in other implementations the model is executed on an external computing device after wireless transmission of the sensor data.

In various implementations, the mouth-wearable sensing device 100 is customized for an individual subject using intraoral characterization data representing the subject's oral anatomy, such as data obtained from an intraoral scan, a three-dimensional digital model of the oral cavity, occlusal analysis data, or combinations thereof, etc. In some implementations, processing the intraoral characterization data includes identifying a first position corresponding to the incisive papilla of the subject and a second position corresponding to a region proximate to a first branch of the greater palatine vasculature of the subject, based on identified bony or surface-geometry landmarks such as palatal contour geometry, the location of the greater palatine foramen or canal as identifiable from radiographic data, or posterior palatal surface contours. In further implementations, processing the intraoral characterization data additionally includes identifying a third position corresponding to a region proximate to a second branch of the greater palatine vasculature, based on identified bony or surface-geometry landmarks, a first molar position corresponding to a location of bite force concentration in a first molar region, and a second molar position corresponding to a location of bite force concentration in a second molar region. The mouth-wearable sensing device 100 is then manufactured such that each sensor is positioned at its corresponding identified position when the device 100 is worn. In further implementations, the mouth-wearable sensing device 100 is formed using additive manufacturing, thermoforming, molding, overmolding, or other suitable fabrication techniques, to produce a body that conforms to the subject's oral anatomy and positions the sensors at the determined locations.

In some embodiments, the greater palatine vasculature is defined as an anatomical region of the posterior hard palate, and sensor placement proximate to that region is determined based on identified bony or surface-geometry landmarks, including one or more of palatal contour geometry, the location of the greater palatine foramen or canal as identifiable from radiographic data, rugae pattern, or posterior palatal surface contours, rather than from direct visualization of vascular branches in surface scan data.

In various implementations, the mouth-wearable sensing device 100 and one or more external devices together form a system for monitoring physiological responses of a subject. In some implementations, the wireless transmitter of circuitry 108 transmits sensor data to one or more external devices, which are configured to receive the sensor data and process it to generate one or more outputs, including visualizations, reports, alerts, or decisions relevant to the physiological state of the subject, etc. In some implementations, the external device is configured to receive sensor data and to process the sensor data using one or more machine learning models. In some implementations, the one or more machine learning models are trained using sensor data including data obtained from subjects experiencing force impact events and baseline physiological data obtained from subjects not experiencing force impact events, etc. In some implementations, the one or more machine learning models are configured to receive as input one or more time-aligned sensor data streams and to output one or more of: a severity metric, a score, a classification, or other quantitative or categorical indication representative of a force impact event experienced by the subject. In further implementations, the external device is configured to use the output of the one or more machine learning models to generate a behavioral recommendation for the subject (e.g., a return-to-play recommendation, a return-to-duty recommendation, or a recommendation to seek medical evaluation, etc.). In still further implementations, the external device is configured to process the sensor data to determine whether the subject has sustained a concussion or other force-impact-related physiological injury.

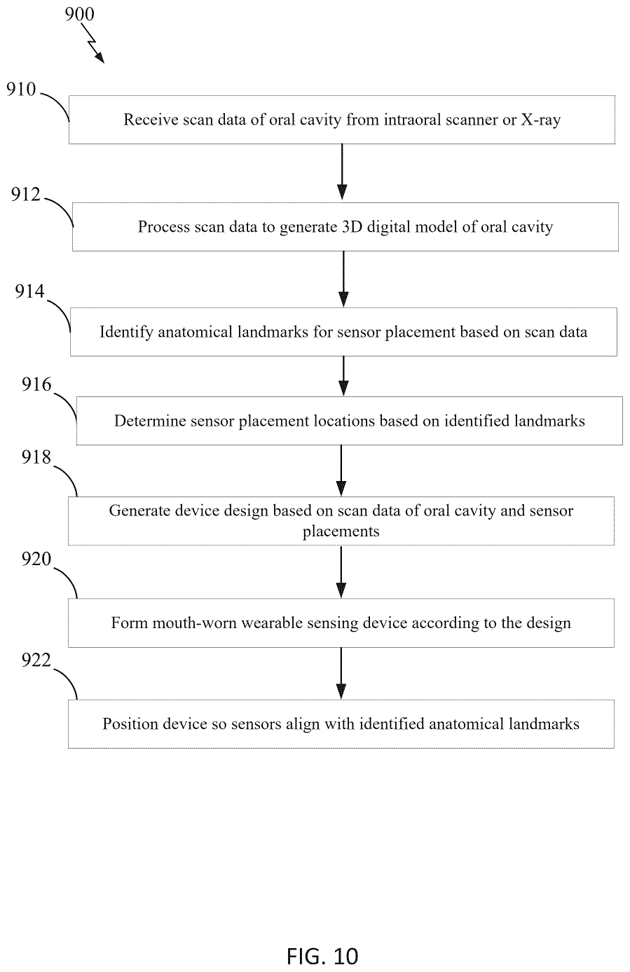

Referring now to FIG. 10, flow diagram is shown for an example process 900 for manufacturing a mouth-wearable sensing device configured to be positioned within an oral cavity of a subject, according to some embodiments. In some implementations, process 900 is performed using one or more computing devices that receive and process intraoral characterization data to produce a three-dimensional digital model of the oral cavity, including anatomical surface data representing the teeth, gum line, and palate. In some implementations, the intraoral characterization data may be obtained using one or more instruments or techniques, including an intraoral surface imaging system and/or a radiographic imaging system, an occlusal analysis system, a bite force measurement device, or combinations thereof, for use in device design and customization. In some embodiments, the intraoral surface imaging system comprises a structured-light scanner, a laser scanner, or an intraoral camera configured to capture overlapping images and/or surface information of intraoral anatomy to generate a three-dimensional surface representation. In some embodiments, the intraoral characterization data further comprises radiographic imaging data acquired using a radiographic imaging system, such as an X-ray imaging system or a cone beam computed tomography (CBCT) system, which may provide cross-sectional or volumetric anatomical data supplementing the three-dimensional surface representation.

At step 910, process 900 includes receiving intraoral characterization data of an oral cavity of the subject. In some implementations, the intraoral characterization data comprises one or more of three-dimensional anatomical surface data of the teeth, gum line, and palate acquired using an intraoral surface imaging system, and occlusal analysis data acquired using an occlusal analysis system or bite force measurement device.

At step 912, process 900 includes processing the intraoral characterization data to generate a three-dimensional digital model of the oral cavity of the subject. In some implementations, the three-dimensional digital model provides a geometric representation of one or more intraoral surfaces, including one or more occlusal surfaces, gingival contours, and palatal contours, suitable for subsequent analysis and/or device design generation.

At step 914, process 900 includes processing the intraoral characterization data and/or the three-dimensional digital model to identify one or more anatomical landmarks of the oral cavity for sensor placement. In some implementations, the anatomical landmarks include one or more structural and surface-geometry landmarks of the palate—such as palatal contour geometry, the location of the greater palatine foramen or canal as identifiable from radiographic data, posterior palatal surface contours, and/or rugae pattern—that serve as proxies for identifying regions proximate to the greater palatine vasculature suitable for sensor placement. In some examples, the anatomical landmarks include the incisive papilla of a subject. In other examples, the landmarks include the greater palatine vasculature of the subject.

At step 916, process 900 includes determining one or more sensor placement locations within the oral cavity based on the identified anatomical landmark(s). In some implementations, determining the sensor placement locations includes selecting one or more positions on the three-dimensional digital model that correspond to the identified bony or surface-geometry landmark(s) such that, when the device is worn, a sensor is positioned proximate to the anatomical region associated with the landmark(s) to improve signal acquisition.

At step 918, process 900 includes generating a design for the mouth-wearable sensing device based on (i) the three-dimensional anatomical surface data of the teeth, gum line, and palate and (ii) the determined sensor placement location(s). In some implementations, the design includes one or more geometric features configured to retain the device on the subject's teeth and/or palate, and further includes one or more cavities, channels, recesses, or mounting regions for supporting the sensors at the determined sensor placement locations. In some examples, the determined sensor placement locations include positions proximate to the incisive papilla and/or positions proximate to the region of the greater palatine vasculature as identified from bony or surface-geometry landmarks.

At step 920, process 900 includes forming the mouth-wearable sensing device according to the design. In some implementations, forming the mouth-wearable sensing device includes fabricating a mouthguard body from one or more biocompatible materials and integrating one or more sensors into the mouthguard body at the sensor placement locations specified by the design. By way of example and not limitation, forming may include additive manufacturing (e.g., three-dimensional printing), thermoforming, molding, overmolding, lamination, and/or other suitable fabrication techniques to produce a customized body that conforms to the subject-specific three-dimensional digital model.

At block 922, process 900 includes forming and/or configuring the device such that, when the mouth-wearable sensing device is positioned in the oral cavity, the sensors align with the identified anatomical landmarks. In some examples, the anatomical landmarks include the incisive papilla of a subject. In other examples, the landmarks include bony or surface-geometry landmarks proximate to the greater palatine vasculature of the subject. In some implementations, the device geometry and sensor mounting regions are selected to align the sensor(s) with the landmark(s) when the device is seated on the subject's teeth and/or palate, thereby supporting consistent placement across repeated uses.

As used herein, the term “proximate,” when used to describe the positional relationship between a sensor and an anatomical landmark, means within a distance sufficient to enable the sensor to obtain a suitable measured signal from or through the anatomical landmark when the mouth-wearable sensing device 100 is worn. What constitutes a suitable measured signal may depend on the sensor type, the target anatomical landmark, and the physiological parameter being measured. For example, a pulse oximeter 140 positioned proximate to the incisive papilla is positioned close enough to the sphenopalatine vasculature to obtain a photoplethysmographic signal of sufficient signal quality for heart rate or oxygen saturation measurement. A thermistor 150 positioned proximate to the greater palatine vasculature is positioned close enough to the palatine vasculature to obtain a temperature measurement representative of core or peripheral body temperature.

Although FIG. 10 depicts blocks 910-922 in a particular order, it should be appreciated that one or more blocks may be omitted, combined, repeated, or performed in a different order depending on the implementation. For example, in some embodiments, identifying the anatomical landmark(s) (block 914) and determining sensor placement location(s) (block 916) may be performed iteratively, including where a candidate placement is adjusted based on manufacturability constraints or fit constraints derived from the three-dimensional digital model.

What is claimed is:

1. A mouth-wearable sensing device to be positioned within an oral cavity of a subject, the mouth-wearable sensing device comprising:
a structure to register against a roof of a mouth of the subject and maintain a fixed placement of a first sensor and a second sensor relative to the roof of the mouth when the mouth-wearable sensing device is worn;
the first sensor supported by the structure and positioned proximate to an incisive papilla of the subject when the mouth-wearable sensing device is worn; and
the second sensor supported by the structure and positioned proximate to a greater palatine vasculature of the subject when the mouth-wearable sensing device is worn.

2. The mouth-wearable sensing device of claim 1, wherein the structure comprises a body shaped to fit over a plurality of teeth of the subject.

3. The mouth-wearable sensing device of claim 2, further comprising:
a first force sensing resistor supported by the structure and positioned in a first molar region of the oral cavity to sense a bite force of the subject when the mouth-wearable sensing device is worn; and
a second force sensing resistor supported by the structure and positioned in a second molar region of the oral cavity to sense the bite force of the subject when the mouth-wearable sensing device is worn.

4. The mouth-wearable sensing device of claim 1, wherein:
the second sensor is positioned proximate to a first branch of the greater palatine vasculature of the subject when the mouth-wearable sensing device is worn;
the first sensor is a pulse oximeter;
the second sensor is a first thermistor; and
the mouth-wearable sensing device further comprises a third sensor supported by the structure and positioned proximate to a second branch of the greater palatine vasculature of the subject when the mouth-wearable sensing device is worn, the third sensor being a second thermistor.

5. The mouth-wearable sensing device of claim 4, further comprising:
a first inertial measurement unit (IMU) mounted on a first sensor assembly with the second sensor, the first sensor assembly positioned on a first side of the oral cavity proximate to the first branch of the greater palatine vasculature when the mouth-wearable sensing device is worn; and
a second inertial measurement unit (IMU) mounted on a second sensor assembly with the third sensor, the second sensor assembly positioned on a second side of the oral cavity proximate to the second branch of the greater palatine vasculature when the mouth-wearable sensing device is worn.

6. The mouth-wearable sensing device of claim 5, further comprising:
a first accelerometer mounted on the first sensor assembly with the second sensor and the first inertial measurement unit; and
a second accelerometer mounted on the second sensor assembly with the third sensor and the second inertial measurement unit.

7. The mouth-wearable sensing device of claim 1, further comprising at least one sensor selected from the group consisting of:
a salivary sensor;
a humidity sensor;
a carbon dioxide (CO2) sensor; and
a chemical sensor array;
wherein the at least one sensor is mounted on a sensor assembly positioned in the oral cavity when the mouth-wearable sensing device is worn.

8. The mouth-wearable sensing device of claim 1, further comprising:
a wireless transmitter to transmit sensor data obtained from one or more sensors of the mouth-wearable sensing device to an external device.

9. The mouth-wearable sensing device of claim 8, further comprising a processor to perform on-device edge processing of the sensor data prior to transmission by the wireless transmitter, wherein the wireless transmitter is further configured to stream the sensor data to a connected external device in real time.

10. A method of manufacturing a mouth-wearable sensing device to be positioned within an oral cavity of a subject, the method comprising:
obtaining intraoral characterization data of the oral cavity of the subject;
identifying, from the intraoral characterization data, a first position corresponding to an incisive papilla of the subject and a second position corresponding to a first branch of a greater palatine vasculature of the subject; and
manufacturing a structure comprising a first sensor and a second sensor, the structure to, when worn, register against a roof of a mouth of the subject and maintain a fixed placement of the first sensor and the second sensor relative to the roof of the mouth,
the first sensor supported by the structure such that, when worn, the first sensor is proximate to the incisive papilla, and
the second sensor supported by the structure such that, when worn, the second sensor is proximate to the first branch of the greater palatine vasculature,
wherein the first sensor and the second sensor are positioned in the structure based on the identified first position and the identified second position.

11. The method of claim 10, further comprising:
identifying, from the intraoral characterization data, a first molar position and a second molar position corresponding to locations of bite force concentration in the oral cavity of the subject; and
manufacturing the structure to further comprise a first force sensing resistor and a second force sensing resistor,
the first force sensing resistor positioned in the structure based on the identified first molar position such that, when worn, the first force sensing resistor is at a first location of bite force concentration in a first molar region to sense a bite force of the subject, and
the second force sensing resistor positioned in the structure based on the identified second molar position such that, when worn, the second force sensing resistor is at a second location of bite force concentration in a second molar region to sense the bite force of the subject.

12. The method of claim 10, further comprising:
identifying, from the intraoral characterization data, a third position corresponding to a second branch of the greater palatine vasculature of the subject; and
manufacturing the structure to further comprise a third sensor positioned in the structure based on the identified third position such that, when worn, the third sensor is proximate to the second branch of the greater palatine vasculature of the subject.

13. The method of claim 12, further comprising:
manufacturing the structure as a mouth guard comprising:
a first inertial measurement unit and a first accelerometer each mounted on a first sensor assembly with the second sensor; and
a second inertial measurement unit and a second accelerometer each mounted on a second sensor assembly with the third sensor.

14. A system comprising:
a mouth guard to be positioned within an oral cavity of a subject, the mouth guard comprising:
a structure to register against a roof of a mouth of the subject and maintain a fixed placement of a first sensor, a second sensor, and a third sensor relative to the roof of the mouth when the mouth guard is worn;
the first sensor supported by the structure and positioned proximate to an incisive papilla of the subject when the mouth guard is worn;
the second sensor supported by the structure and positioned proximate to a first branch of a greater palatine vasculature of the subject when the mouth guard is worn;
the third sensor supported by the structure and positioned proximate to a second branch of the greater palatine vasculature of the subject when the mouth guard is worn; and
a wireless transmitter to transmit sensor data obtained from one or more of the first sensor, the second sensor, and the third sensor to an external device; and
the external device to receive the sensor data from the mouth guard.

15. The system of claim 14, wherein:
the first sensor is a pulse oximeter;
the second sensor is a first thermistor; and
the third sensor is a second thermistor.

16. The system of claim 14, wherein the mouth guard further comprises:
a first inertial measurement unit mounted on a first sensor assembly with the second sensor and positioned proximate to the first branch of the greater palatine vasculature when the mouth guard is worn; and
a second inertial measurement unit mounted on a second sensor assembly with the third sensor and positioned proximate to the second branch of the greater palatine vasculature when the mouth guard is worn.

17. The system of claim 16, wherein the mouth guard further comprises:
a first accelerometer mounted on the first sensor assembly with the second sensor and the first inertial measurement unit; and
a second accelerometer mounted on the second sensor assembly with the third sensor and the second inertial measurement unit.

18. The system of claim 14, wherein the external device is to receive the sensor data and to process the received sensor data to determine whether the subject has sustained a concussion.

19. The system of claim 14, wherein the external device is further configured to:
receive the sensor data from the mouth guard as a time-aligned multi-modal sensor data stream comprising two or more of inertial data, force data, temperature data, and optical physiological data; and
process the multi-modal sensor data stream using one or more trained machine learning models to generate a severity metric representative of a force impact event experienced by the subject and, based on the severity metric, generate a behavioral recommendation for the subject regarding one or more of return-to-play, return-to-duty, or a recommendation to seek further medical evaluation.

20. The system of claim 19, wherein:
the one or more trained machine learning models comprise one or more time-series models including one or more of a convolutional neural network (CNN) or a recurrent neural network (RNN) including a long short-term memory (LSTM) network;
the one or more trained machine learning models are trained on sensor-fusion inputs combining two or more of inertial data, bite-force data, temperature data, and optical physiological data obtained from subjects experiencing force impact events and from subjects not experiencing force impact events;
the multi-modal sensor data stream is time-aligned across sensor modalities; and
the severity metric is generated by detecting patterns and correlations across two or more sensor modalities in the multi-modal sensor data stream that are associated with a force impact event.
